(19) United States
(12) Patent Application Publication
(10) Pub. No.: US 2023/0346681 A1
(43) Pub. Date: Nov. 2, 2023

(54) COMPOSITIONS COMPRISING A POLYPHENOL AND A GLYCEROLATED COMPOUND

(71) Applicant: L'OREAL, Paris (FR)

(72) Inventors: Brady ZARKET, Edison, NJ (US); Anne-Laure Suzanne BERNARD, New York, NY (US); Yixin YANG, Union, NJ (US)

(73) Assignee: L'OREAL, PARIS (FR)

(21) Appl. No.: 17/731,809

(22) Filed: Apr. 28, 2022

(51) Int. Cl. A61K 8/85; A61K 8/34; A61K 8/06; A61Q 1/02

(52) U.S. Cl. CPC A61K 8/85; A61K 8/34; A61K 8/064; A61Q 1/02; A61K 2800/48; A61K 2800/43

(57) ABSTRACT

Compositions for making up the skin are provided. Compositions include: (1) a solvent system comprising (i) water in an amount of at least about 10% by weight with respect to the total weight of the composition; and (ii) at least one C2-C5 monoalcohol in a concentration by weight in the composition that is about 55% or less and in an amount effective to inhibit formation of a precipitate of compound X and compound Y in the composition prior to application; (2) at least one polyphenol X comprising at least two different phenol groups; (3) at least one glycerolated nonionic compound Y and having an HLB of at least about 7; and (4) at least one pigment.

### FIELD OF THE INVENTION

The present invention relates to compositions, in particular foundation compositions for cosmetics, comprising (1) at least one polyphenol X comprising at least two different phenol groups; (2) at least one glycerolated nonionic compound Y, as well as to methods of using said compositions.

### DISCUSSION OF THE BACKGROUND

At the present time on the market for caring for and making up keratin materials, many products claim staying power throughout the day, withstanding external factors such as water, sebum, mechanical friction, etc. (waterproof mascara, food-proof lipsticks, long-lasting foundations). Long-lasting products for the lips, the eyelashes, the eyebrows or the face, which can be used at home, are mainly based on synthetic coating polymers in the presence of organic solvents.

However, consumers, who are increasingly demanding as regards the composition of their cosmetic products, are also seeking to use products with ingredients that are well tolerated such as natural ingredients, with ingredients which have little or no environmental impact and/or ingredients which are compatible with numerous packaging.

The aim of the present invention is to propose compositions which offer excellent staying power of the expected cosmetic effects, notably the color of the makeup on keratin materials (skin, lips, nails, hair, eyelashes, eyebrows) which may extend from one day, including makeup removal at the end of the day, to staying power over several days, which is resistant to mechanical friction, water, sweat and perspiration, sebum, oil, cleansing products such as shower gels, shampoos, two-phase products and certain micellar waters.

There remains a need for improved compositions such as foundation compositions having improved properties with respect to composition ease and/or comfort of application of the composition itself

Accordingly, one aspect of the present invention is a composition, in particular a foundation composition, which has improved properties with respect to composition ease and/or comfort of application of the composition, composition stability, and/or composition color stability, and in particular with respect to improved properties related to the presence of polyphenols such as tannic acid in the compositions including properties such as improved stability and/or water resistance.

### SUMMARY OF THE INVENTION

The present invention relates to compositions, in particular foundation compositions for cosmetics, comprising a solvent system. The solvent system comprises water and the water is present in an amount of at least about 10% by weight with respect to the total weight of the composition. The solvent system further comprises at least one C2-C5 monoalcohol in an amount effective to inhibit formation of a precipitate of compound X and compound Y in the composition prior to application. The compositions further comprise at least one glycerolated nonionic compound Y optionally with a molar mass greater than 200 g/mol. Additionally, the compositions comprise at least one pigment. In certain embodiments, the glycerolated nonionic compound Y has an HLB above 7. In certain embodiments, the at least one C2-C5 monoalcohol is present in the composition in a concentration by weight of about 55% by weight or less. In certain other embodiments, the at least one at least one C2-C5 monoalcohol (e.g., ethanol) may be present in a concentration by weight in the composition ranging from about 5% to about 55%. In certain embodiments, the at least one at least one C2-C5 monoalcohol is or includes ethanol.

According to certain other embodiments the at least one C2-C5 monoalcohol (e.g., ethanol) is present in a concentration by weight in the composition from about 5% to about 55%, the water is present in a concentration by weight in the composition from 10% to about 40%, compound X and compound Y are each independently present in a concentration by weight from about 0.5% to about 25%, and the at least one C2-C5 monoalcohol is present in a concentration such that a weight ratio of (C2-C5 monoalcohol) to (C2-C5 monoalcohol plus water) is present in a weight ratio of at least about 0.3.

The present invention also relates to methods of caring for, and/or making up, and/or priming for caring for and/or making up keratinous material by applying compositions of the present invention to the keratinous material in an amount sufficient to care for, and/or to make up, the keratinous material.

It is to be understood that both the foregoing general description and the following detailed description are exemplary and explanatory only, and are not restrictive of the invention.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 15% of the indicated number (e.g. “about 10%” means 8.5% -11.5% and “about 2%” means 1.7% -2.3%), such as within 10% of the indicated number, such as within 5% of the indicated number.

“A” or “an” as used herein means “at least one.”

“At least one” means one or more and thus includes individual components as well as mixtures/combinations.

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc. Ranges in the form of, for example, “from about 0.5%, 1%, or 2% to about 4%, 5%, or 15%,” also specifically contemplate from about 0.5% to about 4%, from about 0.5% to about 5%, 0.5% to about 15%, from about 1% to about 4%, from about 1% to about 5%, 1% to about 15%, from about 2% to about 4%, from about 2% to about 5%, and 2% to about 15%.

“Film former”, “film-forming polymer” or “film-forming agent” as used herein means a polymer or resin that leaves a film on the substrate to which it is applied, for example, after a solvent accompanying the film former has evaporated, absorbed into and/or dissipated on the substrate. “Additional film former” refer to film formers other than compound X and compound Y, which are capable of forming a film upon removal of the C2-C5 monoalcohol.

“Wax” as used herein is a lipophilic fatty compound that is solid at ambient temperature (25° C.) and changes from the solid to the liquid state reversibly, having a melting temperature of more than 30° C. and, for example, more than 45° C., and a hardness of more than 0.5 MPa at ambient temperature.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C.

“Non-volatile”, as used herein, means having a flash point of greater than about 100° C. “Polymer” as used herein means a compound which is made up of at least two monomers.

“Polymer” as used herein means a compound which is made up of at least two monomers.

“Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. Thus, for example, “free of triethanolamine (TEA)” means that an effective amount (that is, more than trace amounts) of TEA is omitted from the composition, “substantially free of TEA” means that TEA is are present in amounts not greater than 0.1% by weight, and “devoid of TEA” means that TEA is present in amounts not greater than 0.25% by weight, based on the total weight of the composition. The same nomenclature applies for all other ingredients identified throughout the application and in this paragraph such as, for example, oils (compositions of the invention which are “free of oils,” “substantially free of oils,” and “devoid of oils” have meanings consistent with the discussion within this paragraph), even if not specifically discussed for each identified ingredient. Discussed examples of the use of such language are intended to be exemplary, not limiting.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to keratin materials such as lips and evaluating the color of the composition after an extended period of time. For example, the color of a composition may be evaluated immediately following application to keratin materials such as lips and these characteristics may then be re-evaluated and compared after a certain amount of time. Further, these characteristics may be evaluated with respect to other compositions, such as commercially available compositions.

“Keratinous material” or “keratin material” means natural nails, lips, skin such as the face, the body, the hands, and the area around the eyes, and keratin fibres such as head hair, eyelashes, eyebrows, bodily hair of a human, as well as synthetic additions such as false eyelashes, false eyebrows, false nails, etc.

“Physiologically acceptable” means compatible with keratinous material and having a pleasant color, odor and feel, and which does not cause any unacceptable discomfort (stinging or tautness) liable to discourage a consumer from using the composition. Acceptable pH levels for compositions of the present invention are preferably slightly or somewhat acidic, that is, less than 7, preferably 6.5 or less, preferably 6.0 or less, preferably 5.5 or less, including all ranges and subranges therebetween such as, for example 3 to 5, 4 to 6, 3 to 4.5, etc. Further, compositions of the present invention preferably may be in the form of a spray composition such as, for example, a composition having sufficient fluidity to allow it to be suitable for aerosol spraying (preferably further containing propellant) or manual spraying (for example, pump spraying) as is known in the art.

Compositions of the present invention may also be in the form of a gel composition. In this specification, “gel composition” means a composition which does not flow similar to a liquid when applied to a substrate, a composition which has a 3-dimensional network that inhibits the composition from spreading on, or dripping from, a substrate after application owing to gravity. G′ (storage modulus) is higher than G″ (loss modulus) at all range of strains up to 300% strain. Preferably, G′ (storage modulus) is higher than G″ (loss modulus) at low strain, but with G′ decreasing and G″ increasing, the gel may have a gel crossover point at >0.1% strain, preferably >1% strain, and preferably less than 200% strain, preferably less than 150% strain.

“Gel Crossover Point” (Sol/Gel Point), means the point at which the G″ (loss modulus) intersects the G′ (storage modulus), reported in % strain. It is the point at which a composition goes from a more solid state to a more liquid state. An example of a method for determining gel crossover point is as follows: G″ (loss modulus) and G′ (storage modulus) using a Discovery HR-3 Rheometer by TA Instruments, having 40 mm 2° cone-and-plate stainless steel geometry. The test can be run @ 25° C., with test parameter of angular frequency of 1.0 rad/s and logarithmic sweep: Strain % 0.01 to 100.0% or 1000.0% with 10 points per decade. Results reported in % strain.

“Hydrogen bonding interaction” means an interaction involving a hydrogen atom of one of the two reagents and an electronegative heteroatom of the other reagent, such as oxygen, nitrogen, sulfur and fluorine. In the context of the invention, the hydrogen bonding forms between the hydroxyl functions (OH) of the reactive phenol groups of the polyphenol X and the reactive hydroxyl groups of the compound Y, which are capable of forming hydrogen bonding with those of said phenol groups of the polyphenol X.

“Coating agent formed by interaction by hydrogen bonds of at least one polyphenol X comprising at least two different phenol groups with the compound Y” means that the conditions are met so that the reaction can be carried out between the two reagents, in particular that: i) the amount of polyphenol X is sufficient in the composition containing it, and ii) the compound Y is soluble, miscible or solubilized by another solvent in the medium of the composition containing it, and iii) the compound Y has a sufficient number of hydrogen bond acceptor groups to react with the phenol groups of polyphenol X and, in the medium of the composition containing it, and iv) the compound Y, in the medium of the composition containing it, does not comprise in its structure any group which does not allow the formation of hydrogen bonding with the functions of the reactive phenol groups of the polyphenol X, such as for example, one or more anionic group.

“Natural compound” refers to any compound derived directly from a natural substance such as a plant without having undergone any chemical modification.

“Compound of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means 25° C.

“Atmospheric pressure” means 760 mmHg, i.e. 105 pascals.

“Coating agent” refers to any compound which is capable of forming a deposit on the surface of a keratin material to which it has been applied.

“Hydrogen bonding-inhibiting agent” refers to any compound which is capable of preventing hydrogen bonding interaction between the polyphenol X and the compound Y and/or which is capable of dissociating the complex formed by said interaction by breaking the hydrogen bonding.

The compositions and methods of the present invention can comprise, consist of, or consist essentially of the essential elements and limitations of the invention described herein, as well as any additional or optional ingredients, components, or limitations described herein or otherwise useful. For example, the surfactant component of the composition can “consist essentially of” identified surfactant(s) or types of surfactants discussed below.

For purposes of the present invention, the “basic and novel property” associated with compositions, components and methods which “consist essentially of” identified ingredients or actions is “prevention of precipitation or chemical associations generated by compound X and compound Y prior to use.”

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

Polyphenol X

According to the present invention, compositions comprising at least one polyphenol X comprising at least two different phenol groups are provided.

The polyphenols X that may be used according to the present invention include in their structure at least two different phenol groups.

The term “polyphenol” refers to any compound containing in its chemical structure at least two and preferably at least three phenol groups.

The term “phenol group” refers to any group comprising an aromatic ring, preferably a benzene ring, including at least one hydroxyl group (OH).

The term “different phenol groups” refers to phenol groups that are chemically different.

The polyphenols X that may be used according to the invention may be synthetic or natural. They may be in isolated form or contained in a mixture, notably contained in a plant extract. Polyphenols are phenols comprising at least two phenol groups that are differently substituted on the aromatic ring.

The two classes of polyphenols are flavonoids and non-flavonoids.

Examples of flavonoids that may be mentioned include chalcones such as phloretin, phloridzin, aspalathin or neohesperidin; flavanols such as catechin, fisetin, kaempferol, myricetin, quercetin, rutin, procyanidins, proanthocyanidins, pyroanthocyanidins, theaflavins or thearubigins (or thearubrins); dihydroflavonols such as astilbin, dihydroquercetin (taxifolin) or silibinin; flavanones such as hesperidin, neohesperidin, hesperetin, naringenin or naringin; anthocyanins such as cyanidin, delphinidin, malvidin, peonidin or petunidin; catechin tannins such as tannic acid; isoflavonoids such as daidzein or genistein; neoflavanoids; lignans such as pyroresorcinol; and mixtures thereof.

Among the natural polyphenols that may be used according to the invention, mention may also be made of lignins.

Examples of non-flavonoids that may be mentioned include curcuminoids such as curcumin or tetrahydrocurcumin; stilbenoids such as astringin, resveratrol or rhaponticin; aurones such as aureusidin; and mixtures thereof.

As polyphenols that may be used according to the invention, mention may also be made of chlorogenic acid, verbascoside; coumarins substituted with phenols.

According to a particular embodiment of the invention, the polyphenol X will be chosen from catechin tannins such as gallotannins chosen from tannic acid; ellagitannins such as epigallocatechin, epigallocatechin gallate, castalagin, vescalagin, vescalin, castalin, casuarictin, castanopsinins, excoecarianins, grandinin, gradinin, roburins, pterocarinin, acutissimin, tellimagrandins, sanguiin, potentillin, pedunculagin, geraniin, chebulagic acid, repandisinic acid, ascorgeraniin, stachyurin, casuarinin, casuariin, punicacortein, coriariin, cameliatannin, isodeshydrodigalloyl, dehydrodigalloyl, hellinoyl, punicalagin and rhoipteleanins.

According to a particular embodiment of the invention, the polyphenol X is epigallocatechin, in particular a green tea extract having the INCI name Green Tea Extract, notably comprising at least 45% epigallocatechin relative to the total weight of said extract, for instance the commercial product sold under the name Dermofeel Phenon 90 M-C® sold by the company Evonik Nutrition & Care or the commercial product sold under the name Tea Polyphenols Green Tea Extract® by the company Tayo Green Power.

According to a particular embodiment of the invention, the polyphenol X is a procyanidin or a mixture of procyanidins, in particular an extract of maritime pine bark having the INCI name Pinus pinaster Bark/Bud Extract, notably comprising at least 65% by weight of procyanidins relative to the total weight of said extract, such as the commercial product sold under the name Pycnogenol® sold by the company Biolandes Aromes.

Tannic acid will be used more particularly as polyphenol X.

According to a particular embodiment, the polyphenol(s) X according to the invention is preferably present in a content equal or greater than 0.5% by weight, such as from about 0.5%, 0.75% 1%, 1.5% or 2% to about 2.5%, 4%, 5%, 10%, 15%, 20% 25% or 30%—all percentages by weight relative to the total weight of the composition containing it (them).

Nonionic Compound

According to the present invention, compositions comprising at least one glycerolated nonionic compound Y having an HLB of at least about 7. Optionally compound Y will have a molar mass greater than 200 g/mol. In one preferred embodiment, the molar mass of the compound Y is greater than 350 g/mol. According to certain embodiments, the compound Y is surface active (i.e., is a surfactant) and may be capable of depressing surface tension of deionized water when placed in such deionized water at a surfactant concentration of 0.25% by weight at room temperature and pressure to less than about 60 mN/m, such as less than about 50 mN/m, such as less than about 40 mN/m.

For the purposes of the present invention, the term “glycerolated compound” refers to any molecule comprising in its chemical structure a glycerol group or a chain comprising glycerol units —(O -CH₂-CHOH-CH₂)ₘ, where m is 1 or greater. In certain preferred embodiments, the glycerolated compound is a polyglycerolated compound meaning that m is 2 or greater. In this specification, “PG” is used as an abbreviation of polyglycerol or polyglyceryl.

As examples of monoglycerolated or polyglycerolated or polyoxyalkylenated nonionic compounds Y, mention may be made of:
(1) Glycerolated alkyl ethers such as glyceryl lauryl ether,
(2) Polyglycerolated alkyl ether nonionic surfactants, in particular chosen from polyglyceryl-2 oleyl ether and polyglyceryl-4 oleyl ether,
(3) Glycerol or polyglycerol esters of fatty acids, which are optionally polyhydroxylated, in particular chosen from polyglycerol-2 oleate, polyglyceryl-4 caprate, polyglyceryl-6 caprylate, polyglyceryl-3 dicitrate/stearate, polyglyceryl-10 dioleate, polyglyceryl-10 laurate, glyceryl stearate citrate.

Compound Y may also optionally be polyoxyalkenated. For the purposes of the present invention, the term “polyoxyalkenated compound” refers to any molecule comprising in its chemical structure at least chain comprising oxyalkylene units, in particular oxyethylene units—(OCH₂CH₂)n and/or oxypropylene units—(OCH₂CH₂CH₂)ₚ, where n and p are 2 or greater.

Examples of glycerolated compounds that are also polyoxyalkenated include Polyoxyethylenated alkylglycerides, in particular chosen from PEG-6 Caprylic/Capric Glycerides, PEG-60 Almond Glycerides, PEG-10 Olive Glycerides and PEG-45 Palm Kernel Glycerides; PEG-7 Caprylic/capric triglycerides, and PEG-7 Glyceryl cocoate.

According to a preferential embodiment, the compound(s) Y will be chosen from fatty acid esters of polyglycerol, in particular polyglyceryl-2 oleate, polyglyceryl-4 caprate, polyglyceryl-6 caprate, polyglyceryl-10 dioleate; and mixtures thereof.

According to certain embodiments, the glycerolated compounds Y are chosen from those having a single carbon chain or, if having multiple carbon chains, then the multiple carbon chains are unsaturated.

According to a particular embodiment, the compound(s) Y according to the invention is preferably present in a content equal or greater than 0.5% by weight. According to a particular embodiment, the compound(s) Y according to the invention is preferably present in a concentration by weight from about 0.5%, 0.75% 1%, 1.5% or 2% to about 2.5%, 4%, 5%, 10%, 15%, 20% or 30% - all percentages by weight relative to the total weight of the composition containing it (them). According to certain embodiments, the compound Y has on average from two to ten glycerol units per molecule.

According to a preferential embodiment of the invention, the mole ratio of the reactive hydroxyl groups (OH) of the polyphenol(s) X to the hydroxyl groups of the compound(s) Y that are reactive with those of the polyphenol(s) X preferably ranges from 1/3 to 20 (20/1), preferably from 1/2 to 15 (15/1), and preferably from 3/4 to 4 (4/1).

According to certain embodiments the at least one polyphenol X comprising at least two different phenol groups and the at least one glycerolated nonionic compound Y having an HLB of at least about 7 are present in the composition in a X:Y weight ratio from about 1:10 to about 10:1, such as from about 1:5 to about 5:1, such as from about 1:3 to about 3:1, such as from about 1:1 to about 3:1.

C2-C5 Monoalcohol/Water

According to the present invention, compositions comprising at least one C2-C5 monoalcohol are provided. Suitable C2-C5 monoalcohols include ethanol, propanol, butanol, pentanol, isopropanol, isobutanol and isopentanol. Ethanol is particularly preferred.

For use in foundation and other facial makeup, according to certain embodiments, it is desirable to limit the concentration of C2-C5 monoalcohols to less than about 55% by weight in the composition. According to certain embodiments, the concentration of C2-C5 monoalcohols in the composition ranges from about 5%, 15%, 25%, 35% or 40% to about 40%, 45% 50% or 55%.

Furthermore, in certain embodiments, the at least one C2-C5 monoalcohol is present in a concentration by weight that is effective to inhibit formation of a precipitate of compound X and compound Y in the composition prior to application. In order to prevent precipitation, according to certain embodiments, in the solvent system, the C2-C5 monoalcohol(s) is/are present in a weight ratio of (C2-C5 monoalcohol) to (C2-C5 monoalcohol plus water) of at least about 0.3 , such as at least about 0.35, such as at least about 0.4, such as ranging from about 0.3, 0.35, 0.40, 0.45, 0.50, 0.55, 0.60, 0.65, 0.70 or 0.75 to about 0.70, 0.75, 0.80, 0.95, or 0.90. According to certain embodiments, the C2-C5 monoalcohol(s) is/are present in the solvent system from about 0.75 to about 0.90. According to certain other embodiments the remainder of the solvent system is water (i.e., the solvent system is substantially free or free of solvents other than water and C2-C5 monoalcohol.

According to certain embodiments it generally desirable to limit the water to less than about 70% by weight in the composition. According to certain embodiments, the concentration by weight of water in the composition ranges from about 10% to about 40%, 50% or 60%.

In certain embodiments, in the solvent system, the water is present in weight ratio of water to (C2-C5 monoalcohol plus water) from about 0.10, 0.20, or 0.30 to about 0.30, 0.40, 0.50, 0.60 or 0.70.

Further, according to certain embodiments the various ingredients are present such that: the at least one C2-C5 monoalcohol (e.g., ethanol) is present in a concentration by weight in the composition from about 5% to about 55%, the water is present in a concentration by weight in the composition from 10% to about 40%, compound X and compound Y are each independently present in a concentration by weight from about 0.5% to about 25%, and the at least one C2-C5 monoalcohol is present in a concentration such that a weight ratio of (C2-C5 monoalcohol) to (C2-C5 monoalcohol plus water) is present in a weight ratio of at least about 0.3, such as at least about 0.4, such as at least about 0.5, such as at least about 0.6, such as at least about 0.7.

Preferably, the solvent component of the compositions of the present invention consists essentially of, or consists of, water and C2-C5 monoalcohols. Preferably, the solvent component of the composition is “free of,” “devoid of” or “substantially free of” solvents other than water and C2-C5 monoalcohols.

Coloring Agents

According to preferred embodiments of the present invention, compositions optionally further comprising at least one coloring agent are provided.

According to this embodiment, the at least one coloring agent is preferably chosen from pigments, dyes, such as liposoluble dyes, pigments including nacreous pigments, and pearling agents. According to certain preferred embodiments, the composition includes at least one pigment.

Representative liposoluble dyes which may be used according to the present invention include Sudan Red, DC Red 17, DC Green 6, ß-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%, including all ranges and subranges therebetween.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum. Nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride may also be used.

The pigments may be present in a coloring-effective amount. By “coloring-effective amount,” it is meant that the pigments may be present in an amount sufficient to provide visual color, such as when the compositions are applied to the face and observed under standard lighting conditions (e.g., ASTM D1729-2016) on Fitzpatrick skin phototype III. The pigments may be present in the composition in a concentration ranging up to 30% by weight of the total weight of the composition, such as from 2.5% to 20%, and further such as from 5% to 15%, including all ranges and subranges therebetween.

However, it is possible for the compositions of the present invention to be free, substantially free, or devoid of coloring agents as defined above.

Gelling Agent

According to preferred embodiments of the present invention, compositions optionally further comprising at least one gelling agent are provided. Suitable gelling agents include amphiphilic polymers.

The amphiphilic polymers may comprise at least one ethylenically unsaturated monomer containing a sulphonic group, in freeform or partially or totally neutralized form.

The amphiphilic polymers may comprise at least one hydrophobic portion. The hydrophobic portion present in these polymers preferably contains from 6 to 50 carbon atoms, preferably from 6 to 22 carbon atoms, preferably from 6 to 18 carbon atoms and preferably from 12 to 18 carbon atoms, including all ranges and subranges therebetween.

The amphiphilic polymers may have a molar mass ranging from 50,000 g/mole to 10,000,000 g/mole, preferably from 80,000 g/mole to 8,000,000 g/mole, and preferably from 100,000 g/mole to 7,000,000 g/mole.

The amphiphilic polymers may be based on at least one ethylenically unsaturated hydrophilic monomer A and on at least one hydrophobic monomer B. Preferably, the monomer A comprises a strong acid function, in particular a sulphonic acid or phosphonic acid function. The hydrophobic monomer B comprises at least one hydrophobic radical, chosen from: saturated or unsaturated C₆-C₁₈ linear alkyl radicals (for example, n-hexyl, n-octyl, n-decyl, n-hexadecyl, n-dodecyl or oleyl); branched alkyl radicals (for example, isostearic) or cyclic alkyl radicals (for example, cyclododecane or adamantane); C₆-C₁₈ fluoro or alkylfluoro radicals (for example, the group of formula—(CH₂)₂—(CF₂)₉—CF₃); a cholesteryl radical or radicals derived from cholesterol (for example, cholesteryl hexanoate); aromatic polycyclic groups, for instance naphthalene or pyrene; and silicone or alkylsilicone or alkylfluorosilicone radicals. Among these radicals, linear and branched alkyl radicals are preferred.

The amphiphilic polymers may be crosslinked. The crosslinking agents may be chosen from, for example, the polyolefinically unsaturated compounds commonly used for crosslinking polymers obtained by free-radical polymerization. According to one preferred embodiment of the invention, the crosslinking agent is chosen from methylenebisacrylamide, allyl methacrylate or tri methylolpropane triacrylate (TMPTA). The degree of crosslinking preferably ranges from 0.01 mol % to 10 mol %, and preferably from 0.2 mol % to 2 mol %, relative to the polymer, including all ranges and subranges therebetween.

The amphiphilic polymers may be homopolymers or copolymers.

The amphiphilic polymers can be partially or totally neutralized with a mineral base (for example, sodium hydroxide, potassium hydroxide or aqueous ammonia) or an organic base such as monoethanolamine, diethanolamine, triethanolamine, aminomethylpropanediol, N-methylglucamine, or basic amino acids, for instance arginine and lysine, and mixtures thereof.

The amphiphilic polymers may be water-soluble or water-dispersible homopolymers such as, for example, optionally cross-linked polymers of sodium 2-acrylamido-2-methylpropane sulfonate acid such as that used in the commercial product SIMULGEL 800 (CTFA name: Sodium Polyacryloyldimethyl Taurate), cross-linked polymers of ammonium 2-acrylamido-2-methyl propane sulfonate acid (INCI name: AMMONIUM POLYACRYLDIMEHYLTAURAMIDE) such as the product sold under the tradename HOSTACERIN AMPS by Clariant.

The amphiphilic polymers may be chosen from crosslinked or non-crosslinked amphiphilic polymers of 2acrylamido-2-methylpropanesulphonic (AMPS) acid and of at least one ethylenically unsaturated monomer comprising at least one hydrophobic portion containing from 6 to 30 carbon atoms, preferably from 6 to 22 carbon atoms, preferably from 6 to 18 carbon atoms and preferably from 12 to 18 carbon atoms, including all ranges and subranges therebetween.

Suitable examples of amphiphilic polymers include, but are not limited to, hydrophobically-modified sulfonic acid copolymers such as Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant) (crosslinked ethoxylated AMPS/behenyl methacrylate), Ammonium Acryloyldimethyltaurate/Steareth-25 Methacrylate Crosspolymer (Aristoflex HMS) (ethoxylated copolymer of AMPS/stearyl methacrylate crosslinked with trimethylol triacrylate), Aristoflex SNC (crosslinked ethoxylated AMPS/C16-C18), Aristoflex LNC (noncrosslinked AMPS/C12-C14), and mixtures thereof.

According to other embodiments of the present invention, the gelling agent is an acrylic acid polymer, such as a high molecular weight homo or copolymer of acrylic acid that may be crosslinked with a polyalkenyl polyether- e.g., a “carbomer.”

Preferably, if present, the gelling agent(s) is/are present in the compositions of the present invention in amounts ranging from about 0.05 to about 5% by weight, preferably from 0.1 to 2.5% by weight, preferably from 0.3 to 2% and preferably from 0.5 to 1% by weight, all weights based on the weight of the composition as a whole, including all ranges and subranges therebetween such as, for example, 0.1 to 1.5%, 0.25 to 1.25%, 0.4 to 0.75%, etc.

Oil/Fatty Phase

According to embodiments of the present invention, the compositions of the present invention may optionally further comprise at least one oil. “Oil” means any non-aqueous medium which is liquid at ambient temperature (25° C.) and atmospheric pressure (760 mm Hg). Suitable oils can be volatile or non-volatile.

Suitable oils include volatile silicone oils. Examples of such volatile silicone oils include linear or cyclic silicone oils having a viscosity at room temperature less than or equal to 6cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Suitable oils include non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, and for example, the oils sold under the trade names of Isopar or Permethyl. Preferably, the volatile non-silicone oils have a flash point of at least 40° C.

Suitable oils include synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≥10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; pentaerythritol esters; and synthetic ethers containing from 10 to 40 carbon atoms.

If present, the oil(s) is/are present in the compositions of the present invention in an amount ranging from about 0.1% to about 20% by weight, more preferably from about 0.4% to about 15% by weight, and preferably from about 0.5% to about 10% by weight, based on the total weight of the composition, including all ranges and subranges within these ranges.

According to certain embodiments, however, compositions of the present invention are substantially free of, devoid of, or free of oils.

However, in certain embodiments in which one or more oils are included, the composition may be an emulsion. According to other embodiments, the emulsion has external fatty phase and as such may be a water-in-oil (or water-in silicone) emulsion. As one skilled in the art will readily appreciate, The fatty phase generally includes one or more oils, waxes, silicones, and/or other water-insoluble ingredients.

Additional Additives

The composition of the invention can also comprise any additive usually used in the field under consideration. For example, film forming agents, waxes, dispersants such as poly(l2-hydroxystearic acid), sunscreens, preserving agents, fragrances, fillers, neutralizing agents, cosmetic and dermatological active agents, moisturizers, silicone elastomers, chelating agents and mixtures thereof can be added. According to certain embodiments, compositions of the invention include at least one rheology modifier and/or at least one filler.

Rheology modifiers, as one skilled in the art will recognize include ingredients whose function is to modify rheological properties. Such ingredients include natural gums, natural or synthetic polymers, clays, and the like.

Fillers, as one skilled in the art will recognize include ingredients whose function is create bulk, slip or texture and may secondarily modify rheology as well. Such ingredients include, for example, boron nitride, starch powders, talc, mica, rice powders, silicas, and the like.

Additional film-forming agents, as one skilled in the art will recognize include for example silicones such as silicone resins, urethanes, or any of various hydrocarbon film forming polymers such as those including acrylic functionality (ethylenic unsaturation), polyester functionality, vinyl functionality, and the like.

Humectants as one skilled in the art will recognize include for example, polyhydric alcohols, such as glycerin and various glycol such as butylene glycol, hexylene glycol, hydroxyethyl urea and the like.

A non-exhaustive listing of such ingredients can be found in U.S. patent application publication no. 2004/0170586, the entire contents of which is hereby incorporated by reference. Further examples of suitable additional components can be found in the other references which have been incorporated by reference in this application.

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

Needless to say, the composition of the invention should be cosmetically or dermatologically acceptable, i.e., it should contain a non-toxic physiologically acceptable medium and should be able to be applied to the eyelashes of human beings.

Methods of Use

According to preferred embodiments of the present invention, methods of caring for, and/or making up, and/or care for keratinous material by applying compositions of the present invention to the keratinous material in an amount sufficient to care for, and/or to make up, and/or to prime for caring for and/or making up the keratinous material are provided. Preferably, “making up” the keratin material includes applying at least one coloring agent to the keratin material (in either the composition itself or in a color coat compostion applied over the composition) in an amount sufficient to provide color to the keratin material.

In accordance with the preceding preferred embodiments, the compositions of the present invention are applied topically to the desired keratinous material in an amount sufficient to care for, and/or to make up, and/or to prime for caring for and/or making up the keratinous material. The compositions may be applied to the desired area as needed, preferably once or twice daily, more preferably once daily and then preferably allowed to dry before subjecting to contact such as with clothing or other objects (for example, a color coat composition or a topcoat applied over the composition). Preferably, the composition is allowed to dry for about 1 minute or less, more preferably for about 45 seconds or less. The compositions may be used in particular, as a makeup foundation or for other facial makeup (for example, exclusive of the eye area and/or lip area) such as blush, concealer, highlighter, bronzer and the like.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example I—Sample Formulations and Ethanol Tolerance Testing

Sample formulations were prepared by first making a stock solution of eight grams ethanol, 1 gram of water, 1 gram of tannic acid, and 1 gram of a glycerolated nonionic compound Y. Water was sequentially added to the stock solution in order to achieve a monoalcohol/total solvent ratio (in this case, %monoalcohol/(%monoalcohol+%water) that was 0.8. The appearance of turbidity or precipitation was noted. Additional water was then added to achieve an alcohol/total solvent ratio was 0.75. The appearance of turbidity or precipitation was again noted. This was continued to ratios of 0.70 and then additionally in increments of 0.05 until 0.30 was reached. Results were recorded as “clear,” (C) or showing turbidity or a precipitate (X). The results are shown in Tables 1 and 2 below:

TABLE 1
Ethanol Tolerance Testing For Various Compound Y

| | | | PG-2 | | | |
|---|---|---|---|---|---|---|
| | | PG-2 | Di- | PG-3 | PG-6 | |
| Comp. | Ethanol | Ole- | isostea- | Diisostea- | Capry- | PG-6 |
| Y | % | ate | rate | rate | late | Polyricinoleate |
| HLB | | 7 | 4 | 4 | 14 | 3 |
| | 80 | C | C | X | C | X |
| | 75 | C | X | X | C | X |
| | 70 | C | X | X | C | X |
| | 65 | C | X | X | C | X |
| | 60 | C | X | X | C | X |
| | 55 | C | X | X | C | X |
| | 50 | C | X | X | C | X |
| | 45 | X | X | X | C | X |
| | 40 | X | X | X | X | X |
| | 35 | X | X | X | X | X |
| | 30 | X | X | X | X | X |

TABLE 2
Ethanol Tolerance Testing For Various Additional Compounds Y

| | | | PG-4 | | |
|---|---|---|---|---|---|
| | | | Diisostearate/ | | |
| Comp. | Ethanol | PG-4 | Polyhydroxystearate/ | PG-10 | PG-6 |
| Y | % | Caprate | Sebacate | Dioleate | Dicaprate |
| HLB | | 14 | 5 | 11.9 | 10.2 |
| | 80 | C | X | X | C |
| | 75 | C | X | X | C |
| | 70 | C | X | X | X |
| | 65 | C | X | X | X |
| | 60 | C | X | X | X |
| | 55 | C | X | X | X |
| | 50 | C | X | C | X |
| | 45 | X | X | C | X |
| | 40 | X | X | C | X |
| | 35 | X | X | C | X |
| | 30 | X | X | X | X |

### Example II—Sample Formulations and Wear Testing

Sample formulations were prepared by combining and mixing the ingredients below to form various test compositions, each identical except for the polyoglycerolated nonionic compound Y.

Water resistance testing as performed by applying 20 microliters of sample formulation on a dry forearm, followed by spreading the sample evenly with finger over a 3 cm by 3 cm square area. Samples were dried (typically 45 minutes) at ambient conditions, then rubbed with fingers under lukewarm running water. Samples were rubbed down the length of the arm toward the wrist and then backward toward the elbow 10 times (for a total of 20 passes over each sample deposit). Remaining color was compared to the initial color of the deposit, evaluated visually then given a comparative scoring.

Test compositions and water resistance testing results are shown below.

TABLE 3
Water Resistance testing

| | Ex. 1 | Ex. 2 | Ex. 3 |
|---|---|---|---|
| Deionized water | 36.9 | 36.9 | 36.9 |
| Ethanol | 36.9 | 36.9 | 36.9 |
| Tannic Acid | 5 | 5 | 5 |
| PG-4 Caprate | 5 | 0 | 0 |
| PG-6 Caprylate | 0 | 5 | 0 |
| PG-2 Oleate | 0 | 0 | 5 |
| AMPS Polymer | 1.2 | 1.2 | 1.2 |
| Pigment Blend | 15 | 15 | 15 |
| Water | Acceptable | Acceptable | Acceptable |
| resistance | | | |

### Example III—Evaluation of X:Y Ratios (Polyphenol: Polyoxyalkylene Compound) for Ethanol Tolerance.

Five compositions were prepared and evaluated for ethanol tolerance (clarity/turbidity/precipitate) by first mixing tannic acid (5 wt. %), water (52.25 wt. %), and ethanol (42.75 wt. %) to form a first stock solution of tannic acid; and polyglyceryl-6 caprylate (5 wt. %) in water (52.25 wt. %), and ethanol (42.75 wt. %) to form a second stock solution of polyglycerolated compound. The first and second stock solutions were mixed in varying volume ratios (1:3 to 3:1) to form five test compositions such that the test compositions had an approximate weight ratio of X:Y as shown in Table 3, below, which also shows test results. A “C” indicates that all test compositions were clear composition and showed no discernible turbidity or precipitation.

TABLE 3
Ethanol Tolerance Testing

| X:Y | 3:1 | 2:1 | 1:1 | 1:2 | 1:3 |
|---|---|---|---|---|---|
| Ethanol | C | C | C | C | C |
| Tolerance | | | | | |

The results indicate the various ratios of X:Y were able to tolerate ethanol [weight ratio of (C2-C5 monoalcohol) to (C2-C5 monoalcohol plus water) of 0.45].

1. A composition comprising:
(a) a solvent system comprising (i) water in an amount of at least about 10% by weight with respect to the total weight of the composition; and (ii) at least one C2-C5 monoalcohol in a concentration by weight in the composition that is about 55% or less and wherein at least one C2-C5 monoalcohol is present in an amount effective to inhibit formation of a precipitate of compound X and compound Y in the composition prior to application;
(b) at least one polyphenol X comprising at least two different phenol groups
(c) at least one glycerolated nonionic compound Y having an HLB of at least about 7; and (d) at least one pigment.

2. The composition of claim 1, wherein the at least one polyphenol compound X is tannic acid.

3. The composition of claim 1, wherein the at least one C2-C5 monoalcohol is ethanol.

4. The composition of claim 1, wherein the at least one glycerolated nonionic compound Y has on average from two to ten glycerol units per molecule.

5. The composition of claim 1, wherein the composition is an emulsion.

6. The composition of claim 1, wherein the composition is an emulsion having an internal fatty phase.

7. The composition of claim 1, wherein the composition is a water-in-oil emulsion.

8. The composition of claim 1, wherein the at least one C2-C5 monoalcohol is present in the composition in a concentration by weight from about 5% to about 55% by weight.

9. The composition of claim 1, wherein the at least one C2-C5 monoalcohol is present in the composition in a concentration by weight from about 35% to about 55% by weight with respect to the composition.

10. The composition of claim 1, wherein the at least one C2-C5 monoalcohol is present in the composition in a concentration by weight from about 40% to about 50% by weight with respect to the composition.

11. The composition of claim 1, wherein the at least one polyphenol X comprising at least two different phenol groups and the at least one glycerolated nonionic compound Y having an HLB of at least about 7 are present in the composition in a X:Y weight ratio from about from about 1:3 to about 3:1.

12. The composition of claim 1, further comprising an ingredient selected from at least one rheology modifier, at least one filler, at least one additional film-forming agent, at least one humectant, and combinations thereof.

13. The composition of claim 1, wherein the at least one C2-C5 monoalcohol is a C2-C4 monoalcohol.

14. The composition of claim 1 wherein the at least one C2-C5 monoalcohol is ethanol, wherein the ethanol is present in the composition in a concentration by weight from about 35% to about 55% by weight and wherein the composition is an emulsion having an internal fatty phase.

15. The composition claim 1, wherein the at least one C2-C5 monoalcohol is present in a concentration by weight in the composition from about 5% to about 55%, wherein the water is present in a concentration by weight in the composition from 10% to about 40%, wherein compound X and compound Y are each independently present in a concentration by weight from about 0.5% to about 25%, and wherein the at least one C2-C5 monoalcohol is present in a concentration such that a weight ratio of (C2-C5 monoalcohol) to (C2-C5 monoalcohol plus water) in the composition is at least about 0.3.

16. A method of making up keratinous material comprising applying the composition of claim 1 to the keratinous material.
